(19) United States
(12) Patent Application Publication
(10) Pub. No.: US 2020/0061394 A1
(43) Pub. Date: Feb. 27, 2020

(54) PIEZOELECTRIC DRIVING DEVICE, HIGH-INTENSITY FOCUSED ULTRASOUND HANDPIECE DEVICE USING THE SAME, AND CARTRIDGE FOR HANDPIECE DEVICE

(71) Applicant: ILOODA Co., Ltd., Suwon-si (KR)

(72) Inventor: Seon Uk YOO, Osan-si (KR)

(73) Assignee: ILOODA Co., Ltd., Suwon-si (KR)

(21) Appl. No.: 16/270,596

(22) Filed: Feb. 8, 2019

(30) Foreign Application Priority Data
Aug. 24, 2018 (KR) 10-2018-0099117
Aug. 24, 2018 (KR) 10-2018-0099119

(51) Int. Cl. A61N 7/02

(52) U.S. Cl. CPC A61N 7/02; A61N 2007/0034

(57) ABSTRACT

A high-intensity focused ultrasound handpiece (HIFU) device for skin care, includes a handpiece body including a high frequency generator and a controller, which are disposed in the handpiece body, a cartridge detachably coupled to the handpiece body, electrically connected to the handpiece body in a coupled state, and filled with a fluid for generation of ultrasound, the cartridge being provided with a contact head to come into close contact with a skin of a person to be treated, and a piezoelectric driving device dipped in the fluid in the cartridge and provided with a transducer for irradiating the skin with high-intensity focused ultrasound in accordance with a high frequency signal generated from the high frequency generator.

### BACKGROUND OF THE INVENTION

### Field of the Invention

The present invention relates to a handpiece device using high-intensity focused ultrasound (HIFU), which is configured to obtain skin care and anti-aging effects by focusing high-intensity ultrasound energy on a point of a specific region in the skin by a transducer, solidiflying tissue in the specific region using high temperature generated at the focal point and regenerating new skin tissue at the solidified tissue, a piezoelectric driving device used in the handpiece device, and a cartridge used in the handpiece device.

### Description of the Related Art

Generally, a treatment of forming a solidification zone in a dermis layer of the skin, producing collagen in the solidification zone, to fill the solidification zone, and, as such, obtaining skin care effects such as removal of wrinkles is being greatly highlighted. As such a treatment, an invasive method using a microneedle or the like and a non-invasive method using ultrasound or the like are typically used.

For non-invasive treatment, ultrasound is widely used. A medical appliance using high-intensity focused ultrasound (HIFU), which is called an “HIFU device”, has recently been highlighted. For example, such an HIFU device may radiate high-density focused ultrasound into tissue of the skin and, as such, may perform a treatment for skin care such as face lifting or skin tightening in a non-invasive manner.

In most cases, HIFU devices generally and widely used for skin care have common basic configurations. FIG. 1 shows the configuration of a handpiece included in a conventional HIFU device.

The handpiece of the conventional HIFU device shown in FIG. 1 includes a handpiece body 10, and a cartridge 20 as a disposable product detachably coupled to the handpiece body 10.

A transducer 25, which receives ultrasound energy and focuses the received ultrasound energy on a point apart therefrom by a focal distance, is provided at the cartridge 20. A driver including components designated by reference numerals 11, etc. should also be provided to linearly move the transducer 25. In a state in which the cartridge 20 is in contact with the skin, focused ultrasound may be uniformly irradiated onto the contact portion of the skin in accordance with linear movement of the transducer 25 and, as such, a thermal solidification point may be formed in the skin.

As shown in FIG. 1, the driver includes components provided at the handpiece body 10, that is, a linear motor 11 and a driving shaft 12. The driver also includes components provided at the cartridge 20, that is, a connecting shaft 21 connected to the motor driving shaft 12 by magnets 13 or the like, a fixed member 23 fixed to the connecting shaft 21, and a transducer fixing member 24 for fixing the transducer 25 to the fixed member 23.

The cartridge 20 is not configured to be permanently used after coupling thereof, but is configured to be disposed after a certain number of treatment times and, as such, to be replaceable with a new one (furthermore, the focal length of the transducer provided at the cartridge is fixed and, as such, various kinds of cartridges having different focal lengths should be prepared as replaceable cartridges). For this reason, only under the condition that the connecting shaft 21 in the cartridge 20 is connected to the driving shaft 12 in the handpiece body 10 by the magnets 13 or a separate connecting means, can the connecting shaft 21 receive driving force of the linear motor 11, to move the transducer 25.

Meanwhile, the cartridge 20 should be filled with certain liquid in order to enable ultrasound radiation of the transducer 25. In connection with this, a bellows 22 should be separately provided to isolate a power transmission including the connecting shaft 21, etc. in the cartridge 20 from the liquid.

As mentioned above, the conventional HIFU device does not employ a system in which the motor directly drives the transducer disposed in the cartridge, but employs a system in which the motor disposed in the handpiece body indirectly drives the transducer disposed in the cartridge. Although a DC motor or a stepper motor is typically used as a linear motor for accurate control of the transducer, such a motor has a great size and, as such, the cartridge should also have a great size for accommodation of the motor. Furthermore, when the cartridge is scrapped, the motor is scrapped together with the cartridge. With regard to this, the direct drive system in which the motor is disposed in the cartridge is impractical.

However, the above-mentioned configuration of the conventional HIFU device uses the system in which the motor provided at the handpiece body indirectly drives the connecting shaft provided at the cartridge and, as such, may have problems of degraded control accuracy and low stability, as compared to the direct drive system.

Furthermore, since the linear motor moves the driving shaft of the motor forwards and rearwards, spaces for movement of the driving shaft should be secured at front and rear sides of the linear motor. For this reason, it is necessary to increase the length or size of the handpiece body and, as such, there may be a problem of a great limitation as to compactness.

In particular, constituent components having great volumes, such as the linear motor 11, should be disposed in the handpiece body 10 and, as such, it is difficult to dispose constituent components such as an ultrasound generator to supply ultrasound energy irradiated from the transducer 25, a controller, etc. in the handpiece body 10. For this reason, as shown in FIG. 1, the conventional HIFU device has a configuration in which constituent components including an ultrasound generator 53, a controller 52, etc. are disposed in a body 50. In connection with this, the conventional HIFU device inevitably employs a configuration in which transmission of signals is achieved through a cable connecting the handpiece to the body 50. In this case, however, there may be a problem of signal loss occurring during signal transmission through the cable.

In addition, it is necessary to suppress evaporation of moisture as much as possible under the condition that the cartridge is filled with liquid. In the cartridge of the above-mentioned conventional HIFU device, however, considerable moisture loss through the bellows occurs because the cartridge should be provided with the power transmission including the connecting shaft to be connected to the driving shaft of the motor disposed in the handpiece body, etc. As a result, there may be a problem in that a separate sealing structure or means should be provided.

As prior art literature associated with the above-mentioned conventional HIFU device, there are Korean Patent Application Nos. 10-2015-0026533, 10-2017-0013457, and 10-2016-0003984.

### SUMMARY OF THE INVENTION

Therefore, the present invention has been made in view of the above problems, and it is an object of the present invention to provide a piezoelectric driving device capable of achieving very accurate and stable control of a transducer while moving the transducer in a direct driving manner and greatly reducing the size of a handpiece and, as such, achieving compactness of the handpiece, and a high-intensity focused ultrasound (HIFU) handpiece device using the piezoelectric driving device.

In accordance with one aspect of the present invention, the above and other objects can be accomplished by the provision of a high-intensity focused ultrasound handpiece device for skin care including a handpiece body including a high frequency generator disposed in the handpiece body, and a controller disposed in the handpiece body, to control high frequency signal generation of the high frequency generator, a cartridge detachably coupled to the handpiece body, electrically connected to the handpiece body in a coupled state thereof to the handpiece body, and filled with a fluid for generation of ultrasound, the cartridge being provided, at one side thereof, with a contact head to come into close contact with a skin of a person to be treated, and a piezoelectric driving device dipped in the fluid in the cartridge and provided with a transducer for irradiating the skin with high-intensity focused ultrasound in accordance with a high frequency signal generated from the high frequency generator, the piezoelectric driving device moving the transducer by a driving force created by piezoelectric ultrasound generated in accordance with a high frequency signal generated from the high frequency generator.

The cartridge may include a cartridge body formed with an opening allowing the fluid to fill the cartridge body, and provided with the contact head at one side thereof, and a sealing cover coupled to the opening of the cartridge body, to seal an interior of the cartridge body.

The cartridge may further include a printed circuit board (PCB) disposed at an outer surface of the cartridge body and electrically connected to the controller and the high frequency generator when coupled to the handpiece body. The piezoelectric driving device may be disposed in a sealed interior of the cartridge, and is electrically connected to the PCB by electric wires, to receive an associated one of the high frequency signals from the high frequency generator via the PCB.

Alternatively, the cartridge may further include a protection cover coupled to the cartridge body or the sealing cover, and a PCB disposed in the protection cover and provided with a connector protruding from the protection cover, to provide electrical connection of the PCB to the handpiece body in a coupled state thereof to the handpiece body. The PCB may be electrically connected to the piezoelectric driving device by sealed electric wires, thereby transmitting, to the piezoelectric driving device, an associated one of the high frequency signals generated from the high frequency generator disposed in the handpiece body.

The piezoelectric driving device may include a driving frame fixed to one side of the cartridge within the cartridge, a piezoelectric driving unit coupled to the driving frame, to generate ultrasound vibration by the piezoelectric sound generated in accordance with an associated one of the high frequency signals from the high frequency generator, and a piezoelectric operating unit coupled with the transducer. The piezoelectric operating unit may be moved by use of the ultrasound vibration generated from the piezoelectric driving unit as a driving force, thereby moving the transducer.

The piezoelectric driving unit may include a piezoelectric motor for generating ultrasound, and a piezoelectric driving shaft connected to the piezoelectric motor at one end thereof, to generate vibration by the ultrasound from the piezoelectric motor, while being fixed to one side of the driving frame at the other end thereof. The piezoelectric operating unit may be coupled to the piezoelectric driving shaft such that the piezoelectric operating unit moves along the piezoelectric driving shaft in accordance with the vibration of the piezoelectric driving shaft, thereby moving the transducer coupled thereto.

The piezoelectric operating unit may include an operating body, a transducer coupling member provided at one side of the operating body and coupled with the transducer, and a driving core member provided at a core receiving portion formed at the operating body, to surround the piezoelectric driving shaft, the driving core member moving along the piezoelectric driving shaft in accordance with the vibration of the piezoelectric driving shaft.

The driving frame may include first and second guide shafts spaced apart from each other in a movement direction of the piezoelectric operating unit. The piezoelectric operating unit may include an operating body, a transducer coupling member provided at the operating body and coupled with the transducer, a driving core member surrounding the piezoelectric driving shaft while being received in a core receiving portion formed at the operating body, the driving core member moving along the piezoelectric driving shaft in accordance with the vibration of the piezoelectric driving shaft, a first slide groove provided at one side of the operating body while being open at one side thereof, to allow the first guide shaft to be fitted therein, and a second slide groove provided at the other side of the operating body while being open at one side thereof, to allow the second guide shaft to be fitted therein, and, as such, the first and second slide grooves may move along the first and second guide shafts in a slide-guided manner, respectively, in accordance with movement of the operating body along the piezoelectric driving shaft.

The cartridge may further include a PCB disposed at an outer surface of the cartridge body and electrically connected to the controller and the high frequency generator when coupled to the handpiece body. The piezoelectric operating unit may include a column member provided at one side of the piezoelectric operating unit, to move along a guide groove formed at the driving frame in accordance with the movement of the piezoelectric operating unit, and a magnet provided at an end of the column member. The PCB may include a plurality of Hall sensors provided at an area facing the magnet while being uniformly spaced from one another by a predetermined distance, to sense movement of the magnet according to the movement of the piezoelectric operating unit. Accordingly, the movement of the piezoelectric operating unit may be sensed through the plurality of Hall sensors.

The handpiece body may further include a battery receiving unit for receiving a battery therein and, as such, operation of the piezoelectric driving device in the cartridge according to generation of an associated one of the high frequency signals from the high frequency generator and transmission of the generated high frequency signal under control of the controller is carried out in accordance with supply of electric power from the battery, thereby enabling driving of the transducer to generate high-intensity focused ultrasound and to radiate the generated high-intensity focused ultrasound.

The handpiece body may further include an operating unit to allow a person, to be treated, to perform manipulation for driving of the handpiece device, and a display unit for displaying information as to a driving state of the handpiece device.

In accordance with another aspect of the present invention, there is provided a piezoelectric driving device provided at a cartridge of a high-intensity focused ultrasound handpiece device for skin care including a driving frame dipped in a fluid contained in a body of the cartridge and fixed to the body of the cartridge at one side thereof, a piezoelectric driving unit coupled to the driving frame, to generate ultrasonic vibration by ultrasound generated in accordance with a high frequency signal, a transducer for irradiating a skin with high-intensity focused ultrasound in accordance with a high frequency signal, and a piezoelectric operating unit coupled with the transducer. The piezoelectric operating unit may be moved by use of the ultrasound vibration generated from the piezoelectric driving unit as a driving force, thereby moving the transducer.

The driving frame may include a frame body, a driving unit coupling/support member provided at one side of the frame body, and an operation support member provided at the other side of the frame body. The piezoelectric driving unit may include a piezoelectric motor for generating piezoelectric ultrasound in accordance with a high frequency signal from the high frequency generator, a driving unit coupling body coupled to the driving unit coupling/support member, the driving unit coupling body receiving the piezoelectric motor therein, and a piezoelectric driving shaft connected to the piezoelectric motor at one end thereof, to generate vibration by the piezoelectric ultrasound from the piezoelectric motor, while being fixed to one side of the driving frame at the other end thereof in accordance with the coupling of the driving unit coupling body to the driving unit coupling/support member. The piezoelectric operating unit may be coupled to the piezoelectric driving shaft such that the piezoelectric operating unit moves along the piezoelectric driving shaft in accordance with the vibration of the piezoelectric driving shaft, thereby moving the transducer coupled thereto.

The piezoelectric operating unit may include an operating body, a transducer coupling member provided at the operating body and coupled with the transducer, and a driving core member provided at a core receiving portion formed at the operating body, to surround the piezoelectric driving shaft. The driving core member may move along the piezoelectric driving shaft in accordance with the vibration of the piezoelectric driving shaft.

The driving core member may include a first driving core provided at one side of the core receiving portion of the operating body and provided with a groove corresponding to the piezoelectric driving shaft, a second driving core provided at the other side of the core receiving portion, to face the first driving core, and provided with a groove corresponding to the piezoelectric driving shaft, and an elastic support ring for elastically supporting a state in which the piezoelectric driving shaft is fitted in a hole formed by the groove of the first driving core and the groove of the second driving core.

The driving frame may include first and second guide shafts spaced apart from each other in a movement direction of the piezoelectric operating unit. The piezoelectric operating unit may include a first slide groove provided at one side of the operating body while being open at one side thereof, to allow the first guide shaft to be fitted therein, and a second slide groove provided at the other side of the operating body while being open at one side thereof, to allow the second guide shaft to be fitted therein, and, as such, the first and second slide grooves move along the first and second guide shafts in a slide-guided manner, respectively, in accordance with movement of the operating body along the piezoelectric driving shaft.

In accordance with another aspect of the present invention, there is provided a cartridge detachably coupled to a handpiece body of a high-intensity focused ultrasound handpiece device for skin care including a cartridge body filled with a fluid for generation of ultrasound and provided, at one side thereof, with a contact head to come into close contact with a skin of a person to be treated, a sealing cover coupled to an opening of the cartridge body, to seal an interior of the cartridge body, and a piezoelectric driving device dipped in the fluid being contained in an interior of the cartridge body sealed by the cartridge body and the sealing cover, and provided with a transducer for irradiating the skin with high-intensity focused ultrasound in accordance with a high frequency signal, the piezoelectric driving device moving the transducer by a driving force created by piezoelectric ultrasound, wherein, when the cartridge is electrically connected to the handpiece body in accordance with coupling thereof to the handpiece body, the transducer is driven by the piezoelectric driving device in the sealed interior of the cartridge body.

The cartridge may further include a protection cover coupled to the cartridge body or the sealing cover, and a PCB disposed in the protection cover and provided with a connector protruding from the protection cover, to provide electrical connection of the PCB to the handpiece body in a coupled state thereof to the handpiece body. The PCB may be electrically connected to the piezoelectric driving device by sealed electric wires, thereby transmitting, to the piezoelectric driving device, a high frequency signal received from a high frequency generator disposed in the handpiece body.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing a configuration of a conventional high-intensity focused ultrasound (HIFU) device;

FIG. 2A is a perspective view illustrating an HIFU handpiece device according to an embodiment of the present invention;

FIG. 2B is an exploded perspective view illustrating the HIFU handpiece device according to the illustrated embodiment of the present invention;

FIG. 3 is a perspective view illustrating a wireless system configuration of the HIFU handpiece device according to the illustrated embodiment of the present invention;

FIG. 4 is a schematic view illustrating an inner configuration of the handpiece device illustrated in FIG. 3;

FIG. 5 is a perspective view illustrating a cartridge of the HIFU handpiece device according to the illustrated embodiment of the present invention;

FIG. 6 is an exploded perspective view of the cartridge illustrated in FIG. 5;

FIG. 7 is an enlarged view illustrating an inner configuration of the cartridge illustrated in FIG. 5;

FIG. 8 is a perspective view illustrating a piezoelectric driving device disposed in the cartridge in accordance with the illustrated embodiment of the present invention;

FIG. 9 is an exploded perspective view illustrating a piezoelectric driving unit of the piezoelectric driving device illustrated in FIG. 8;

FIG. 10 is an exploded perspective view illustrating a piezoelectric operating unit of the piezoelectric driving device illustrated in FIG. 9; and

FIG. 11 is a view explaining operation of the piezoelectric driving device ac cording to the illustrated embodiment of the present invention and operation of the HIFU handpiece device using the piezoelectric driving device.

### DESCRIPTION OF THE PREFERRED EMBODIMENTS

Hereinafter, concrete contents of a high-intensity focused ultrasound (HIFU) handpiece device according to the present invention and a cartridge detachably coupled to a HIFU handpiece used in the HIFU handpiece device will be described with reference to the accompanying drawings.

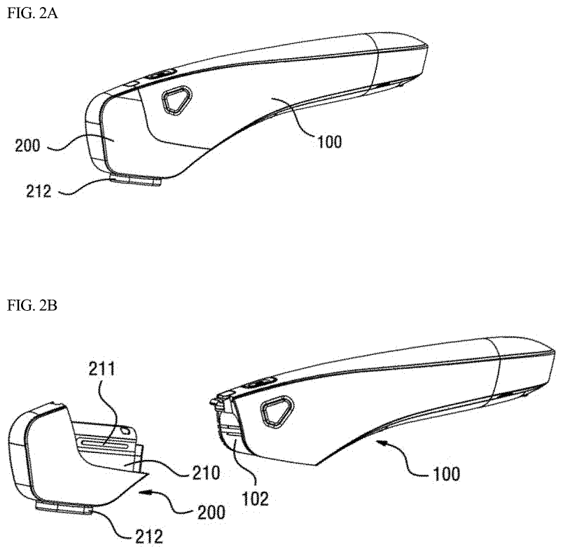

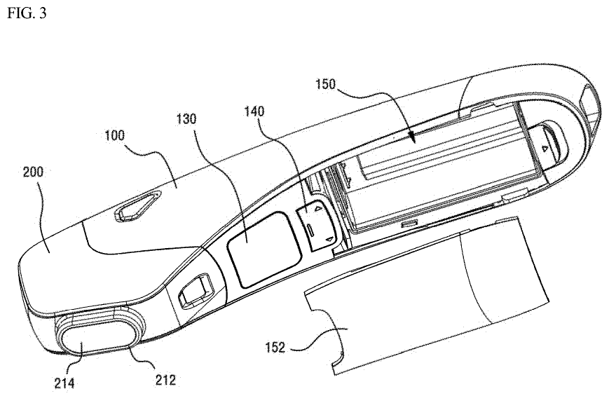

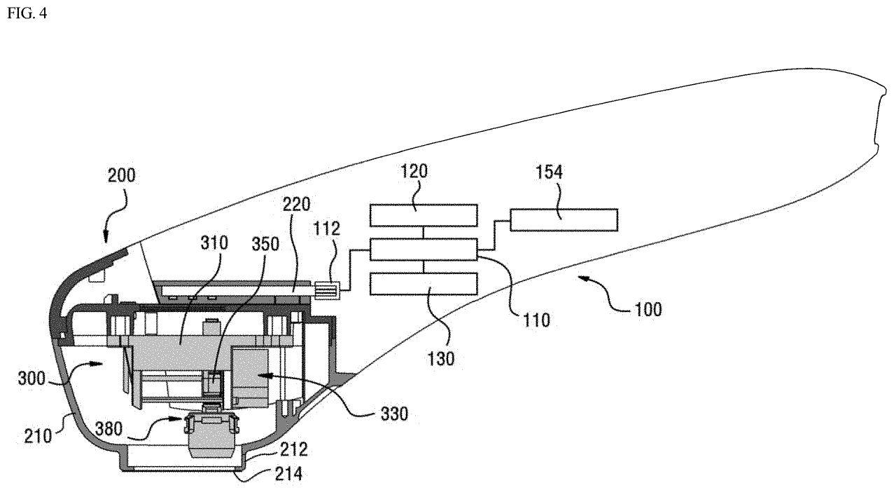

First, an HIFU handpiece device according to an embodiment of the present invention will be described with reference to FIGS. 2A to 4. FIGS. 2A and 2B are perspective views of the HIFU handpiece device according to the illustrated embodiment of the present invention. FIG. 3 is a perspective view illustrating a wireless system configuration of the HIFU handpiece device according to the illustrated embodiment of the present invention. FIG. 4 is a schematic view illustrating an inner configuration of a handpiece body illustrated in FIG. 3.

The HIFU handpiece device according to the illustrated embodiment of the present invention may be configured to be connected to an appliance body, as in the conventional HIFU device. However, the present invention has a feature in that constituent components such as an ultrasound generator and a controller are not provided at the appliance body, differently from the conventional HIFU device, but are disposed in the HIFU handpiece device according to the illustrated embodiment, nevertheless, the HIFU handpiece device according to the illustrated embodiment achieves compactness. Furthermore, the present invention has a feature in that the handpiece device may be configured to receive electric power from a battery disposed therein without receiving electric power from the appliance body and, as such, may be embodied as a wireless type.

In the present invention, the appliance body may function to supply electric power to the handpiece device when the handpiece device is wired, and may be used as a device for recharging the handpiece device when the handpiece device is wireless.

As illustrated in FIGS. 2A to 4, the HIFU handpiece device according to the illustrated embodiment of the present invention includes a handpiece body 100, a cartridge 200, and a piezoelectric driver (300) disposed in the cartridge 200.

FIG. 2A shows a state of the HIFU handpiece device according to the illustrated embodiment in which the cartridge 200 is coupled to the handpiece body 100. FIG. 2B shows a state in which the cartridge 200 is separated from the handpiece body 100.

The handpiece device may be configured to be connected to a separate appliance body (not shown) in a wired manner and to operate in a wireless manner without being connected to the separate appliance body.

In FIGS. 2A and 2B, reference numeral “212” designates a contact head which is a portion of the cartridge 200 to contact the skin of a person to be treated (hereinafter simply referred to as a “patient”), reference numeral “210” designates a body of the cartridge 200, reference numeral “211” designates a handpiece coupling section, and reference numeral “102” designates a cartridge coupling section.

As illustrated in FIGS. 2A and 2B, when the handpiece coupling section 211, which is provided at the cartridge body 210 of the cartridge 200, is coupled to the cartridge coupling section 102, which is provided at a front portion of the handpiece body 100, a printed circuit board (PCB) provided at the cartridge 200 is electrically connected to a controller provided at the handpiece body 100 and, as such, a transducer disposed in the cartridge 200 may perform irradiation with ultrasound while moving under control of the controller.

FIG. 3 is a perspective view showing a lower surface of the HIFU handpiece device illustrated in FIGS. 2A and 2B. As illustrated in FIG. 3, the HIFU handpiece device according to the illustrated embodiment of the present invention may include an operating unit to allow the patient to perform manipulation for driving of the handpiece device, a display unit 130 for displaying information as to a driving state of the handpiece device, and a battery receiving unit 150 for receiving a battery therein.

The handpiece of the conventional HIFU device cannot achieve compactness when the above-described configuration including the operating unit, the display unit and the battery receiving unit is embodied in the handpiece, because the driving motor installed in the handpiece, such as a linear motor, occupies a considerable space. However, the present invention has a feature in that a linear motor is eliminated, and, in place, a piezoelectric driving device is disposed in the cartridge, and, as such, the handpiece may achieve compactness while accommodating constituent components such as an ultrasound generator, a controller, an operating unit, a display unit, and a battery receiving unit therein.

The HIFU handpiece device according to the illustrated embodiment of the present invention has no connection configuration, except for electrical connection between the cartridge 200 and the handpiece body 100. That is, in the HIFU handpiece device, there is no power connection configuration as in the configuration of the conventional HIFU device in which the shaft of the cartridge is connected to the driving shaft of the driving device.

As illustrated in FIG. 4, the handpiece body 100 includes an ultrasound generator 120 disposed in the handpiece body 100, and a controller 110 disposed in the handpiece body 100, to control generation of an ultrasound signal from the ultrasound generator 120. The handpiece body 100 may further include a display 130 and a battery 154.

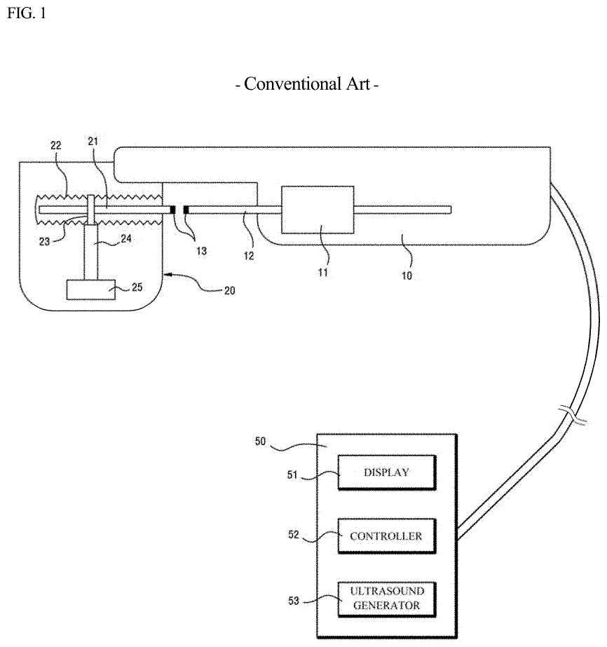

In the conventional HIFU device, a linear motor and a motor driving shaft are disposed in a handpiece body, and a control board to control the linear motor is also provided at the handpiece body, and, as such, it may be impossible to dispose a further constituent component in the handpiece body (FIG. 1). In practice, the linear motor and the motor driving shaft occupy most of the handpiece and, as such, constituent components such as an ultrasound generator for generating a signal for creation of ultrasound to be transmitted to a transducer and a controller cannot be provided at the handpiece body, but are inevitably provided at an appliance body connected to the handpiece (For this reason, the handpiece and the appliance body are connected in a wired manner in the conventional HIFU device.).

In the handpiece device according to the present invention, however, constituent components such as a linear motor and a motor driving shaft are not disposed in the handpiece body 100 and, as such, considerable extra space may be secured in the handpiece body 100. Accordingly, the ultrasound generator 120 and the controller 110 for controlling the ultrasound generator 120, which are difficult to provide at the handpiece of the conventional HIFU device (thus, inevitably being provided at the appliance body) may be installed in the handpiece body 100, and, as such, the handpiece device according to the present invention has a feature in that the size of the appliance body may be greatly reduced or the appliance body itself may be eliminated, and, at the same time, the handpiece may be further compacted.

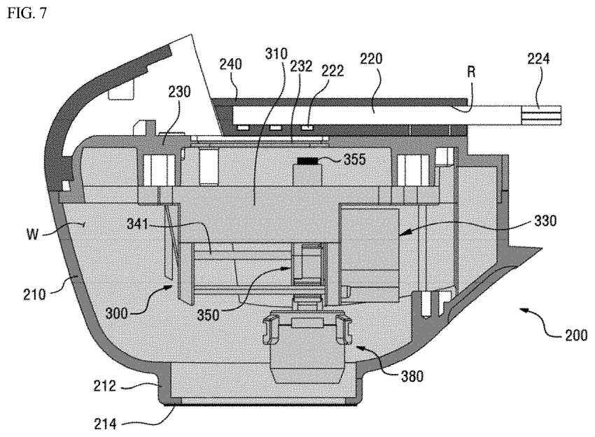

As illustrated in FIGS. 4 and 7, the cartridge 200 detectably coupled to the handpiece body 100 in the HIFU handpiece device according to the illustrated embodiment of the present invention includes the cartridge body 210, which is filled with a fluid W for generation of ultrasound, and the contact head 212, which is to closely contact the skin of a patient. The contact head 212 is disposed at one side of the cartridge body 210. The cartridge 200 also includes a PCB 220 disposed at an outer surface of the cartridge body 210.

The piezoelectric driving device 300, which functions as a driving means for driving a transducer 380, to move the transducer 380, is disposed in the cartridge body 210, together with the transducer 380. The piezoelectric driving device 300 and the transducer 380 are dipped in the fluid W filling the cartridge body 210.

The cartridge 200 is configured to substantially completely seal the interior thereof in order to prevent the fluid W from being lost due to evaporation or the like. In connection with this, the cartridge of the conventional HIFU device has a configuration in which fluid loss may easily occur, because the cartridge has a power connection configuration to be connected to the motor provided at the handpiece body. On the contrary, the cartridge 200 of the handpiece device according to the illustrated embodiment of the present invention has a feature in that the interior of the cartridge 200 is substantially completely sealed and, as such, there is no or little substantial fluid loss of the cartridge 200.

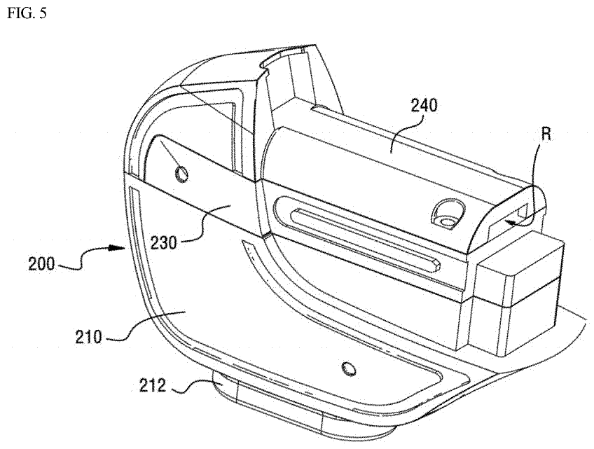

More concrete contents as to the configuration of the cartridge according to the illustrated embodiment of the present invention will be described with reference to FIGS. 5 to 7.

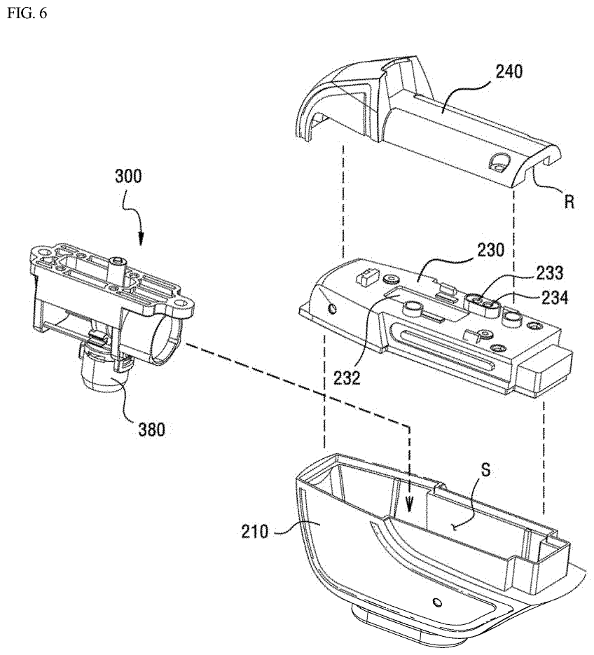

FIG. 5 illustrates the overall configuration of the cartridge according to the illustrated embodiment of the present invention. FIG. 6 is an exploded perspective view of the cartridge configuration illustrated in FIG. 5. FIG. 7 illustrates an inner configuration of the cartridge illustrated in FIG. 5.

As illustrated in FIGS. 5 to 7, the cartridge 200 according to the illustrated embodiment of the present invention may include, in addition to the cartridge body 210, a sealing cover 230 and a protection cover 240. In the cartridge body 210, the piezoelectric driving device 300, which drives the transducer for movement of the transducer, is disposed.

The cartridge body 210 is filled therein with the fluid W for generation of ultrasound. The contact head 212, which is to closely contact the skin of a patient, is provided at one side of the cartridge body 210. The cartridge body 210 is formed with an opening at a top thereof.

The sealing cover 230 is coupled to the opening of the cartridge body 210, to seal the interior of the cartridge body 210.

The piezoelectric driving device 300 is dipped in the fluid W contained in the cartridge body 210 and sealed by the cartridge body 210 and the sealing cover 230. The piezoelectric driving device 300 includes the transducer 380, which irradiates the skin with high-intensity focused ultrasound via the contact head 212 in accordance with a high frequency signal. The piezoelectric driving device 300 drives the transducer 380 by driving force created by piezoelectric ultrasound, for movement of the transducer 380.

As illustrated in FIGS. 5 to 7, the protection cover 240 is configured to be coupled to an upper end of the sealing cover 230. Alternatively, the protection cover 240 may be configured to be coupled to one side of the cartridge body 210.

A connector 224 may protrude from the protection cover 240 in order to achieve an electrical connection when the cartridge 200 is coupled to the handpiece. The protection cover 240 may be provided with a seat groove R for receiving the PCB 220. The PCB 220 is electrically connected to the piezoelectric driving device 300 via sealed electric wires, to transmit a high frequency signal from the high frequency generator disposed in the handpiece body 200 to the piezoelectric driving device 300.

That is, the cartridge 200 according to the present invention may be embodied in such a manner that the fluid-filled inner space of the cartridge 200 is sealed by the cartridge body 210 and the sealing cover 230, and the protection cover 240 is disposed outside the sealing cover 230, to protect the PCB 220.

The sealing cover 230 may be provided with a window 232 such that the piezoelectric driving device 300 disposed in the cartridge body 210 and the PCB 220 provided at the protection cover 240 face each other through the window 232.

A magnet 355 is provided at one side of a piezoelectric operating unit 350 included in the piezoelectric driving device 300. On the other hand, Hall sensors 222 are provided at one side of the PCB 220, to face the magnet 355. Accordingly, the Hall sensors 222 may sense movement of the transducer 380 according to operation of the piezoelectric driving device 300 via the magnet 355. This configuration will be more concretely described later.

Although the cartridge 200 is configured to be completely sealed by the cartridge body 210 and the sealing cover 230, there may be an occasion that the fluid contained in the cartridge 200 leaks slightly through, for example, a film-attached portion of the contact head 212. When such an occasion becomes severe, the fluid may be lost to an extent deteriorating the reliability of ultrasound radiation.

To this end, a fluid supplement hole 233 may be provided at the sealing cover 230 in order to allow the user to supplement the fluid into the sealed space defined by the cartridge body 210 and the sealing cover 230 (for example, to supplement the fluid using an injector or the like) when the above-described fluid leakage has occurred. In this case, an air vent hole 234 may also be provided to vent air present in the cartridge body 210.

The fluid supplement hole 233 and the air vent hole 234 may be formed at a single protrusion provided at the sealing cover 230, to be arranged in parallel. The protrusion, in detail, the fluid supplement hole 233 and the air vent hole 234, is sealed by a separate film or the like and, as such, the interior of the cartridge 200 may be completely sealed.

Although, in the case of FIGS. 5 to 7, the cartridge body 210 and the sealing cover 230 have been described as being coupled to constitute a single sealed body, the present invention is not limited thereto. The cartridge of the present invention may also have a configuration in which the cartridge body 210 and the sealing cover 230 are integrally formed to form a single body.

Meanwhile, a concrete configuration of the piezoelectric driving device 300 disposed in the cartridge according to the illustrated embodiment of the present invention will be described with reference to FIGS. 7 to 9.

As illustrated in FIG. 7, the piezoelectric driving device according to the illustrated embodiment of the present invention uses a piezoelectric motor as a driving unit in order to enable the piezoelectric driving device to operate in a state of being dipped in the fluid W contained in the cartridge body 210.

The piezoelectric motor is also referred to as an “ultrasound motor” and has an advantage in that the piezoelectric motor uses a considerably low driving voltage, as compared to conventional linear motors or DC motors, while being manufactured to have a very small size.

Such a piezoelectric motor has also been used in conventional cases. However, the inventors have developed a piezoelectric driving device capable of exhibiting suitable performance in an HIFU device through use of a piezoelectric motor, after conducting active research into application of a piezoelectric motor to an HIFU device. The present invention provides a piezoelectric driving device capable of accurately controlling movement of a transducer through an operation according to driving force of a piezoelectric motor, even though the piezoelectric driving device is dipped in a fluid contained in a cartridge body.

Accordingly, the piezoelectric driving device according to the illustrated embodiment of the present invention has features in that it may be possible to achieve very accurate and stable control through movement of the transducer carried out in a direct driving manner within the cartridge, it is unnecessary to provide a driving unit for driving the transducer at the handpiece body because constituent components of the driving unit are disposed in a sealed state in the cartridge, and it may be possible to achieve low noise, high speed, and low-voltage design by virtue of provision of the piezoelectric motor.

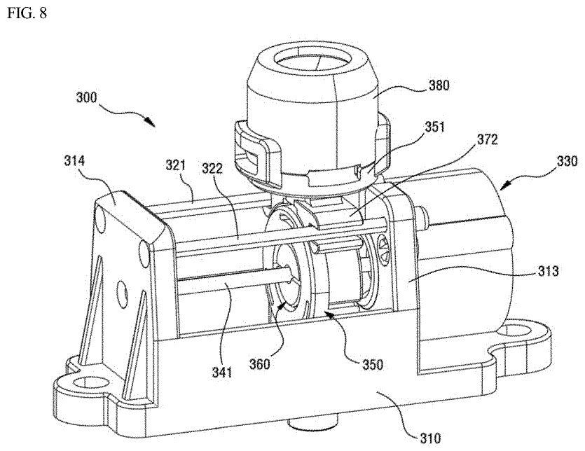

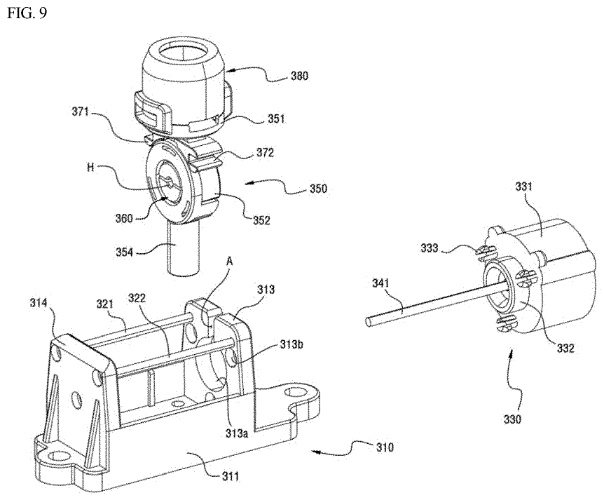

As illustrated in FIGS. 8 and 9, the piezoelectric driving device 300 includes a driving frame 310 fixed to one side of the cartridge body 210 within the cartridge body 210, a piezoelectric driving unit 330 for generating driving force using ultrasound generated in accordance with a high frequency signal from the high frequency generator 120, and a piezoelectric operating unit 350 for moving the transducer 380 while being moved by the driving force generated from the piezoelectric driving unit 330.

The piezoelectric operating unit 350, to which the transducer 380 is coupled, moves along a piezoelectric driving shaft 341 of the piezoelectric driving unit 330 in accordance with vibration of the piezoelectric driving shaft 341.

As illustrated in FIGS. 8 and 9, the driving frame 310 may include a frame body 311, a driving unit coupling/support member 313 provided at one side of the frame body 311, and an operation support member 314 provided at the other side of the frame body 311. The driving frame 310 may also include first and second guide shafts 321 and 322, each of which has one end fixed to the driving unit coupling/support member 313 and the other end fixed to the operation support member 314.

As illustrated in FIG. 9, a body coupling hole 313a and engagement holes 313b may be provided at the driving unit coupling/support member 313. The piezoelectric driving unit 330 is primarily coupled to the body coupling hole 313a, and is secondarily engaged with the engagement holes 313b. Accordingly, the piezoelectric driving unit 330 may be firmly coupled to the driving unit coupling/support member 313.

The piezoelectric driving member 330 includes a piezoelectric motor (not shown) for generating piezoelectric ultrasound in accordance with a high frequency signal from the ultrasound generator 120 (FIG. 4) provided at the handpiece body, and a driving unit coupling body 331 coupled to the driving unit coupling/support member 313 while accommodating the piezoelectric motor. The piezoelectric motor is received in the driving unit coupling body 331 and, as such, is not visible in FIGS. 8 and 9.

As the piezoelectric motor of the piezoelectric driving unit 330 generates ultrasound vibration in a state in which the piezoelectric driving unit 330 is coupled to the driving unit coupling/support member 313, the vibration is transmitted to the entirety of the driving frame 310. In connection with this, an intermediate space A is formed at a portion of the driving unit coupling/support member 313, to more or less attenuate vibration transmitted to the entirety of the driving frame 310. It may also be possible to more or less absorb vibration transmitted to the driving frame 310 by providing a member made of a vibration absorbing material at the intermediate space A.

The piezoelectric motor disposed in the driving unit coupling body 331 may be provided with a piezoelectric driving shaft 314. The piezoelectric driving shaft 314 is connected, at one end thereof, to the piezoelectric motor and, as such, generates vibration in accordance with piezoelectric ultrasound from the piezoelectric motor. When the driving unit coupling body 331 is coupled to the driving unit coupling/support member 313, the other end of the piezoelectric driving shaft 314 is fixed to the operation support member 314.

A body coupling portion 332 and slide-fit engagement portions 333 are provided at one side of the driving unit coupling body 331. The body coupling portion 332 may be coupled to the body coupling hole 313a of the driving unit coupling/support member 313 in a tight fit manner. At the same time, the slide-fit engagement portions 333 may be firmly engaged with the engagement holes 313b in a slide-fit manner.

Meanwhile, as illustrated in FIGS. 8 and 9, the piezoelectric operating unit 350 is coupled to the piezoelectric driving shaft 341 such that the piezoelectric operating unit 350 is movable along the piezoelectric driving shaft 341 in accordance with vibration of the piezoelectric driving shaft 341 and, as such, the transducer 380 coupled to the piezoelectric operating unit 350 is movable.

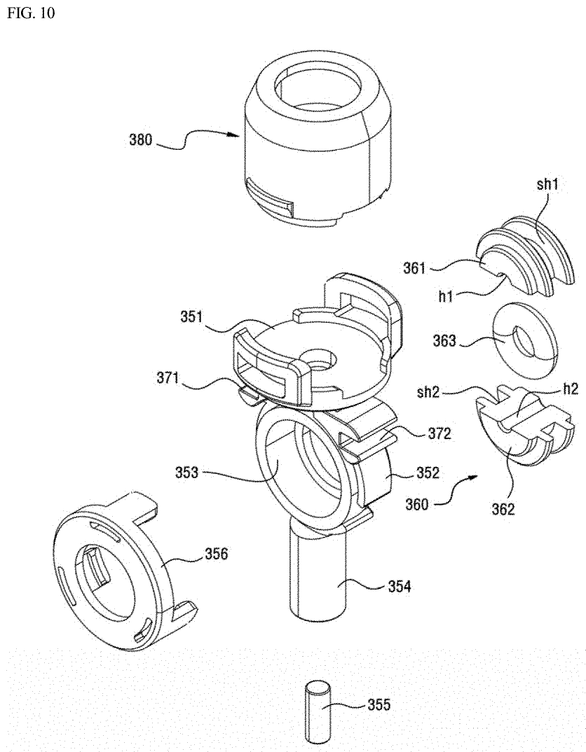

As illustrated in FIGS. 9 and 10, the piezoelectric operating unit 350 may include an operating body 352, a transducer coupling member 351 provided at the operating body 352 and coupled with the transducer 380, and a driving core member 360 surrounding the piezoelectric driving shaft 341 while being received in a core receiving portion 353 formed at the operating body 352. The driving core member 360 is movable along the piezoelectric driving shaft 341 in accordance with vibration of the piezoelectric driving shaft 341.

The transducer coupling member 351 may be disposed at one end of the operating body 352 and, as such, the transducer 380 may be coupled to the operating body 352. A column member 354 may be provided at the other end of the operating body 352, to receive the magnet 355 as described above.

The driving core member 360 disposed in the core receiving portion 353 of the piezoelectric operating unit 350 may include a first driving core 361 disposed in one side of the core receiving portion 353 of the operating body 352 and provided with a groove h1 corresponding to the piezoelectric driving shaft 341, and a second driving core 362 disposed in the other side of the core receiving portion 353, to face the first driving core 361, and provided with a groove h2 corresponding to the piezoelectric driving shaft 341. The driving core member 360 may also include an elastic support ring 363 for elastically supporting a state in which the piezoelectric driving shaft 341 is fitted in a hole H formed by the groove h1 of the first driving core 361 and the groove h2 of the second driving core 362.

The first and second driving cores 361 and 362 may be made of specific metal. The piezoelectric driving shaft 341 may extend through the hole H formed by the grooves h1 and h2 of the first and second driving cores 361 and 362 without being tightly fitted in the hole H, that is, under the condition in which a micro-gap is formed between the piezoelectric driving shaft 341 and the hole H.

As a micro-gap is present between the hole H and the piezoelectric driving shaft 341, ultrasound vibration generated from the piezoelectric driving shaft 341 is transmitted to the driving core unit 360 and, as such, the piezoelectric operating unit 350 may move in accordance with the transmitted vibration.

In this case, the movement speed of the piezoelectric operating unit 350 may be adjusted as the frequency of a frequency signal generated from the high frequency generator 120 is controlled by the controller 110 (FIG. 3).

Vibration is more rapidly generated at a higher frequency, thereby causing the piezoelectric operating unit 350 to move more rapidly. On the other hand, at a lower frequency, the piezoelectric operating unit 350 moves more slowly.

The controller 110 may control the transducer to move at an appropriate speed by appropriately controlling the frequency of the frequency signal in accordance with operation of the user to manipulate the device or sensing of a position of the transducer.

As illustrated in FIG. 10, a first support groove sh1 is formed at the first driving core 361, and a second support groove sh2 is formed at the second driving core 362. The elastic support ring 363 is received in the first and second support grooves sh1 and sh2 under the condition that the first and second driving cores 361 and 362 are in contact with each other to form the hole H and, as such, the elastic support ring 363 elastically supports' the contact state of the first and second driving cores 361 and 362.

As elastic support of the first and second driving cores 361 and 362 is achieved by the elastic support ring 363, as described above, vibration of the piezoelectric driving shaft 341 may be reliably transmitted to the first and second driving cores 361 and 362 and, as such, the piezoelectric operating unit 350 may be easily movable.

In addition, as illustrated in FIG. 10, a core support cover 356 may be provided to support a state in which the driving core unit 360 is received in the core receiving portion 353 of the operating body 352 in the piezoelectric operating unit 350. When the core support cover 356 is coupled to the operating body 352 in a state in which the driving core unit 360 is received in the core receiving portion 353, the core support cover 356 supports the received state of the driving core unit 360.

Meanwhile, when movement of the piezoelectric operating unit 350 is achieved only by the piezoelectric driving shaft 341 and the driving core unit 360, stable movement of the piezoelectric operating unit 350 may not be obtained due to characteristics of the piezoelectric driving shaft 341 and the driving core unit 360.

To this end, as illustrated in FIGS. 8 to 10, the piezoelectric driving device according to the illustrated embodiment of the present invention may include a first slide groove 371 provided at one side of the operating body 352 of the piezoelectric operating unit 350 and a second slide groove 372 at the other side of the operating body 352. The first slide groove 371 is fitted around the first guide shaft 321, and the second slide groove 372 is fitted around the second guide shaft 372. When the piezoelectric operating unit 350 is moved by the piezoelectric driving shaft 341 and the driving core unit 360, the piezoelectric operating unit 350 is guided by the first guide shaft 321 fitted in the first slide groove 371 and the second guide shaft 322 fitted in the second slide groove 372 and, as such, stable movement of the piezoelectric operating unit 350 may be achieved.

If the piezoelectric operating unit 350 moves in a state in which the first and second guide shafts 321 and 322 are fitted in holes, respectively, in place of the first and second slide grooves 371 and 372, there may be a problem in that movement of the piezoelectric operating unit 350 may be inefficiently carried out due to friction generated between each of the guide shafts 321 and 322 and the associated hole. To this end, in the piezoelectric driving device according to the illustrated embodiment of the present invention, each of the first and second slide grooves 371 and 372 in the piezoelectric operating unit 350 is formed to be open at one side and, as such, to substantially have a 90°-rotated U shape. As the guide shafts 321 and 322 are fitted in the first and second slide grooves 371 and 372 formed as described above, respectively, there may be a feature in that movement of the piezoelectric operating unit 350 is smoothly and stably guided without friction.

As illustrated in FIG. 7, the above-described piezoelectric driving device 300 is configured to be driven in the fluid W contained in the cartridge body 210. In connection with this, the cartridge body 210 is configured to be substantially sealed in order to prevent loss of the fluid W contained therein.

As illustrated in FIG. 7, the PCB 220 is disposed at an outer top surface of the cartridge body 210. The connector 224 is provided at an exposed end of the PCB 220.

When the cartridge 200 is coupled to the handpiece body 100, the connector 224 is coupled to a contact 112 provided at the handpiece body 100 and, as such, electrical connection may be achieved.

The piezoelectric driving device 300 is disposed in the cartridge body 210 in a fixed state. The PCB 220, which is disposed adjacent to the piezoelectric driving device 300 outside the cartridge body 210, is electrically connected to the piezoelectric driving device 300 via electric wires. In this case, the electric wires may be treated by a sealing process in order to maintain the interior of the cartridge body 210 in a sealed state.

The piezoelectric driving unit 330 of the piezoelectric driving device 300 and the transducer 380 are electrically connected to the PCB 220 by sealed electric wires and, as such, are connected to the controller 110 disposed in the handpiece body 100 when the connector 224 is connected to the contact 112 in accordance with coupling of the cartridge 200 to the handpiece body 100. In this state, the controller 110 may transmit a high frequency signal for generation of high-intensity focused ultrasound to be irradiated by the transducer 380 and a high frequency signal for generation of ultrasound vibration as driving force of the piezoelectric driving unit 330 while controlling the high frequency generator 120. The PCB 220 may transfer respective signals to the transducer 380 and the piezoelectric driving unit 330 via associated ones of the electric wires.

As high frequency signals generated from the high frequency generator 120 are transmitted to the transducer 380 and the piezoelectric driving unit 330, respectively, under control of the controller 110, as described above, the transducer 380 transmits ultrasound energy to a specific position in the skin through radiation of high-intensity focused ultrasound according to a focal length of a piezoelectric ceramic disposed in the transducer 380, and, at the same time, the piezoelectric driving unit 330 generates ultrasound vibration in accordance with the associated high frequency signal and, as such, moves the piezoelectric operating unit 350, thereby causing the transducer 380 to move.

In this case, the contact head 212 of the cartridge 200 is open at a bottom portion thereof, and the open portion is sealed by a film 214 made of a specific material. When the skin of a patient is subjected to ultrasound treatment, ultrasound energy radiated through the transducer 380 passes through the film 214 under the condition that the portion of the contact head 212 corresponding to the film 214 is in contact with the skin, and is then transferred to tissue present at a focal distance in the skin.

Meanwhile, as illustrated in FIG. 7, the magnet 355 is fixed to an end of the piezoelectric operating unit 350 in the piezoelectric driving device 300, and magnetic field sensors such as the Hall sensors 222 provided at the PCB 220 are arranged in plural at an area facing the magnet 355 while being uniformly spaced apart from one another by a predetermined distance. As the piezoelectric operating unit 350 moves the transducer 380 in accordance with driving force of the piezoelectric driving unit 330, the magnet 355 moves. At this time, the Hall sensors 222 on the PCB 220 sense a magnetic field of the magnet 355 and, as such, sense and trace movement of the piezoelectric operating unit 350, that is, movement of the transducer 380 (a signal sensed by each Hall sensor 222 is transmitted to the controller 110 and, as such, the controller 110 obtains information as to sensed and traced movement of the transducer 380, and controls generation of a high frequency signal based on the obtained information).

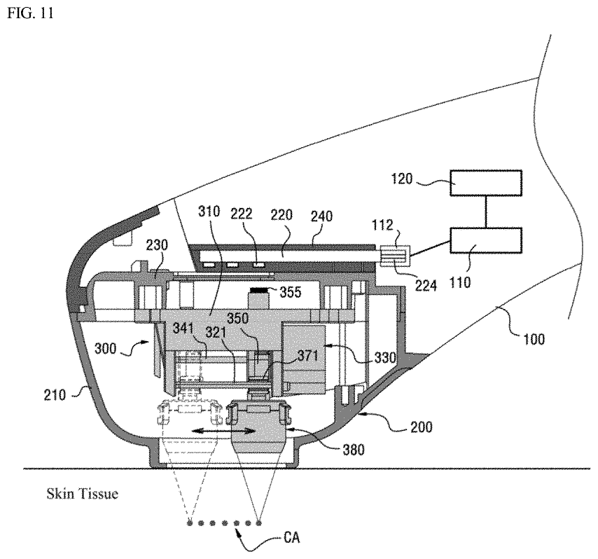

Hereinafter, operation of the cartridge having the above-described configuration according to the illustrated embodiment of the present invention and operation of the handpiece in the HIFU handpiece device including the cartridge will be described with reference to FIG. 11.

The transducer 380 coupled to the piezoelectric driving device 300 disposed in the cartridge 200 is a kernel component of the HIFU device. The focal distance of ultrasound varies in accordance with the curvature and installation position of the piezoelectric ceramic disposed in the transducer 380 and, as such, the treatment depth of the skin tissue (treatment area or position) by the ultrasound varies.

Once the piezoelectric ceramic is installed in the transducer 380, the focal length of the piezoelectric ceramic is fixed and, as such, the treatment depth is fixed. The kind of the piezoelectric ceramic (the curvature of the piezoelectric ceramic) and the installation position of the piezoelectric ceramic are determined in accordance with which one of an SMAS layer, a muscle layer and a dermis layer in the skin is determined as a treatment area. Accordingly, cartridges respectively provided with transducers suitable for different treatment areas are prepared and, as such, a selected one of the cartridges meeting a selected treatment area may be used under the condition that the selected cartridge is coupled to the handpiece body.

Information as to the focal length of the transducer or the treatment depth (information previously determined in accordance with the installation position of the piezoelectric ceramic in the transducer) is stored in a memory (not shown) provided at the PCB 220. Information as to the frequency of a high frequency signal for generation of ultrasound to be radiated through the transducer and information as to the frequency of a high frequency signal for driving of the piezoelectric motor may also be previously stored in the memory.

Accordingly, when the cartridge 200 is coupled to the handpiece body 100, the connector 224 is connected to the contact 112 and, as such, electrical connection between the cartridge 200 and the handpiece body 100 is achieved. In this state, the above-described information stored in the memory of the PCB 220 is transmitted to the controller 110 of the handpiece body 100. Accordingly, the controller 110 controls signals generated from the high frequency generator 120 in accordance with the transmitted information and, as such, controls generation of ultrasound from the transducer 380 and the piezoelectric motor.

When a thermal solidification point is formed at a predetermined depth in accordance with ultrasound energy generated through transmission of a high frequency signal to the transducer 380 under control of the controller 110 under the condition that the contact head 212 of the cartridge 200 is closely in contact with the skin, the controller 110 controls a high frequency signal transmitted to the piezoelectric driving unit 330, to move the piezoelectric operating unit 350 and, as such, to move the transducer 380. In accordance with movement of the transducer 380, a thermal solidification point is formed at the next position in the same manner as described above.

In such a manner, a plurality of uniformly spaced thermal solidification points CA is created in a treatment area in the skin tissue and, as such, treatment is completed. Collagen is produced or tightening is achieved at the thermal solidification points during a self-healing procedure after the treatment and, as such, skin lifting effects may be obtained.

As apparent from the above description, the present invention provides a piezoelectric driving device capable of achieving very accurate and stable control of a transducer while moving the transducer in a direct driving manner and greatly reducing the size of a handpiece and, as such, achieving compactness of the handpiece and achieving low noise, high speed, and low-voltage design, an HIFU handpiece device using the piezoelectric driving device, and a cartridge used in the handpiece device.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A high-intensity focused ultrasound handpiece device for skin care comprising:
a handpiece body comprising a high frequency generator disposed in the handpiece body, and a controller disposed in the handpiece body, to control high frequency signal generation of the high frequency generator;
a cartridge detachably coupled to the handpiece body, electrically connected to the handpiece body in a coupled state thereof to the handpiece body, and filled with a fluid for generation of ultrasound, the cartridge being provided, at one side thereof, with a contact head to come into close contact with a skin of a person to be treated; and
a piezoelectric driving device dipped in the fluid in the cartridge and provided with a transducer for irradiating the skin with high-intensity focused ultrasound in accordance with a high frequency signal generated from the high frequency generator, the piezoelectric driving device moving the transducer by a driving force created by piezoelectric ultrasound generated in accordance with a high frequency signal generated from the high frequency generator.

2. The high-intensity focused ultrasound handpiece device according to claim 1, wherein the cartridge comprises:
a cartridge body formed with an opening allowing the fluid to fill the cartridge body, and provided with the contact head at one side thereof; and
a sealing cover coupled to the opening of the cartridge body, to seal an interior of the cartridge body.

3. The high-intensity focused ultrasound handpiece device according to claim 1, wherein:
the cartridge further comprises a printed circuit board (PCB) disposed at an outer surface of the cartridge body and electrically connected to the controller and the high frequency generator when coupled to the handpiece body; and
the piezoelectric driving device is disposed in a sealed interior of the cartridge, and is electrically connected to the PCB by electric wires, to receive an associated one of the high frequency signals from the high frequency generator via the PCB.

4. The high-intensity focused ultrasound handpiece device according to claim 2, wherein the cartridge further comprises:
a protection cover coupled to the cartridge body or the sealing cover; and
a printed circuit board (PCB) disposed in the protection cover and provided with a connector protruding from the protection cover, to provide electrical connection of the PCB to the handpiece body in a coupled state thereof to the handpiece body, the PCB being electrically connected to the piezoelectric driving device by sealed electric wires, thereby transmitting, to the piezoelectric driving device, an associated one of the high frequency signals generated from the high frequency generator disposed in the handpiece body.

5. The high-intensity focused ultrasound handpiece device according to claim 1, wherein the piezoelectric driving device comprises:
a driving frame fixed to one side of the cartridge within the cartridge;
a piezoelectric driving unit coupled to the driving frame, to generate ultrasound vibration by the piezoelectric sound generated in accordance with an associated one of the high frequency signals from the high frequency generator; and
a piezoelectric operating unit coupled with the transducer, the piezoelectric operating unit being moved by use of the ultrasound vibration generated from the piezoelectric driving unit as a driving force, thereby moving the transducer.

6. The high-intensity focused ultrasound handpiece device according to claim 5, wherein:
the piezoelectric driving unit comprises a piezoelectric motor for generating ultrasound, and a piezoelectric driving shaft connected to the piezoelectric motor at one end thereof, to generate vibration by the ultrasound from the piezoelectric motor, while being fixed to one side of the driving frame at the other end thereof; and
the piezoelectric operating unit is coupled to the piezoelectric driving shaft such that the piezoelectric operating unit moves along the piezoelectric driving shaft in accordance with the vibration of the piezoelectric driving shaft, thereby moving the transducer coupled thereto.

7. The high-intensity focused ultrasound handpiece device according to claim 6, wherein the piezoelectric operating unit comprises an operating body, a transducer coupling member provided at one side of the operating body and coupled with the transducer, and a driving core member provided at a core receiving portion formed at the operating body, to surround the piezoelectric driving shaft, the driving core member moving along the piezoelectric driving shaft in accordance with the vibration of the piezoelectric driving shaft.

8. The high-intensity focused ultrasound handpiece device according to claim 6, wherein:
the driving frame comprises first and second guide shafts spaced apart from each other in a movement direction of the piezoelectric operating unit; and
the piezoelectric operating unit comprises an operating body, a transducer coupling member provided at the operating body and coupled with the transducer, a driving core member surrounding the piezoelectric driving shaft while being received in a core receiving portion formed at the operating body, the driving core member moving along the piezoelectric driving shaft in accordance with the vibration of the piezoelectric driving shaft, a first slide groove provided at one side of the operating body while being open at one side thereof, to allow the first guide shaft to be fitted therein, and a second slide groove provided at the other side of the operating body while being open at one side thereof, to allow the second guide shaft to be fitted therein, whereby the first and second slide grooves move along the first and second guide shafts in a slide-guided manner, respectively, in accordance with movement of the operating body along the piezoelectric driving shaft.

9. The high-intensity focused ultrasound handpiece device according to claim 6, wherein:
the cartridge further comprises a printed circuit board (PCB) disposed at an outer surface of the cartridge body and electrically connected to the controller and the high frequency generator when coupled to the handpiece body;
the piezoelectric operating unit comprises a column member provided at one side of the piezoelectric operating unit, to move along a guide groove formed at the driving frame in accordance with the movement of the piezoelectric operating unit, and a magnet provided at an end of the column member; and
the PCB comprises a plurality of Hall sensors provided at an area facing the magnet while being uniformly spaced from one another by a predetermined distance, to sense movement of the magnet according to the movement of the piezoelectric operating unit,
whereby the movement of the piezoelectric operating unit is sensed through the plurality of Hall sensors.

10. The high-intensity focused ultrasound handpiece device according to claim 1, wherein the handpiece body further comprises a battery receiving unit for receiving a battery therein, whereby operation of the piezoelectric driving device in the cartridge according to generation of an associated one of the high frequency signals from the high frequency generator and transmission of the generated high frequency signal under control of the controller is carried out in accordance with supply of electric power from the battery, thereby enabling driving of the transducer to generate high-intensity focused ultrasound and to radiate the generated high-intensity focused ultrasound.

11. The high-intensity focused ultrasound handpiece device according to claim 10, wherein the handpiece body further comprises an operating unit to allow a person, to be treated, to perform manipulation for driving of the handpiece device, and a display unit for displaying information as to a driving state of the handpiece device.

12. A piezoelectric driving device provided at a cartridge of a high-intensity focused ultrasound handpiece device for skin care comprising:
a driving frame dipped in a fluid contained in a body of the cartridge and fixed to the body of the cartridge at one side thereof,
a piezoelectric driving unit coupled to the driving frame, to generate ultrasonic vibration by ultrasound generated in accordance with a high frequency signal;
a transducer for irradiating a skin with high-intensity focused ultrasound in accordance with a high frequency signal; and
a piezoelectric operating unit coupled with the transducer, the piezoelectric operating unit being moved by use of the ultrasound vibration generated from the piezoelectric driving unit as a driving force, thereby moving the transducer.

13. The piezoelectric driving device according to claim 12, wherein:
the driving frame comprises a frame body, a driving unit coupling/support member provided at one side of the frame body, and an operation support member provided at the other side of the frame body;
the piezoelectric driving unit comprises a piezoelectric motor for generating piezoelectric ultrasound in accordance with a high frequency signal from the high frequency generator, a driving unit coupling body coupled to the driving unit coupling/support member, the driving unit coupling body receiving the piezoelectric motor therein, and a piezoelectric driving shaft connected to the piezoelectric motor at one end thereof, to generate vibration by the piezoelectric ultrasound from the piezoelectric motor, while being fixed to one side of the driving frame at the other end thereof in accordance with the coupling of the driving unit coupling body to the driving unit coupling/support member; and
the piezoelectric operating unit is coupled to the piezoelectric driving shaft such that the piezoelectric operating unit moves along the piezoelectric driving shaft in accordance with the vibration of the piezoelectric driving shaft, thereby moving the transducer coupled thereto.

14. The piezoelectric driving device according to claim 13, wherein the piezoelectric operating unit comprises an operating body, a transducer coupling member provided at the operating body and coupled with the transducer, and a driving core member provided at a core receiving portion formed at the operating body, to surround the piezoelectric driving shaft, the driving core member moving along the piezoelectric driving shaft in accordance with the vibration of the piezoelectric driving shaft.

15. The piezoelectric driving device according to claim 14, wherein the driving core member comprises a first driving core provided at one side of the core receiving portion of the operating body and provided with a groove corresponding to the piezoelectric driving shaft, a second driving core provided at the other side of the core receiving portion, to face the first driving core, and provided with a groove corresponding to the piezoelectric driving shaft, and an elastic support ring for elastically supporting a state in which the piezoelectric driving shaft is fitted in a hole formed by the groove of the first driving core and the groove of the second driving core.

16. The piezoelectric driving device according to claim 15, wherein:
the driving frame comprises first and second guide shafts spaced apart from each other in a movement direction of the piezoelectric operating unit; and
the piezoelectric operating unit comprises a first slide groove provided at one side of the operating body while being open at one side thereof, to allow the first guide shaft to be fitted therein, and a second slide groove provided at the other side of the operating body while being open at one side thereof, to allow the second guide shaft to be fitted therein, whereby the first and second slide grooves move along the first and second guide shafts in a slide-guided manner, respectively, in accordance with movement of the operating body along the piezoelectric driving shaft.

17. A cartridge detachably coupled to a handpiece body of a high-intensity focused ultrasound handpiece device for skin care comprising:
a cartridge body filled with a fluid for generation of ultrasound and provided, at one side thereof, with a contact head to come into close contact with a skin of a person to be treated;
a sealing cover coupled to an opening of the cartridge body, to seal an interior of the cartridge body; and
a piezoelectric driving device dipped in the fluid being contained in an interior of the cartridge body sealed by the cartridge body and the sealing cover, and provided with a transducer for irradiating the skin with high-intensity focused ultrasound in accordance with a high frequency signal, the piezoelectric driving device moving the transducer by a driving force created by piezoelectric ultrasound,
wherein, when the cartridge is electrically connected to the handpiece body in accordance with coupling thereof to the handpiece body, the transducer is driven by the piezoelectric driving device in the sealed interior of the cartridge body.

18. The cartridge according to claim 17, further comprising:
a protection cover coupled to the cartridge body or the sealing cover; and
a printed circuit board (PCB) disposed in the protection cover and provided with a connector protruding from the protection cover, to provide electrical connection of the PCB to the handpiece body in a coupled state thereof to the handpiece body, the PCB being electrically connected to the piezoelectric driving device by sealed electric wires, thereby transmitting, to the piezoelectric driving device, a high frequency signal received from a high frequency generator disposed in the handpiece body.
